(19) United States
(12) Patent Application Publication
(10) Pub. No.: US 2016/0362393 A1
(43) Pub. Date: Dec. 15, 2016

(54) ENDOPARASITE CONTROL AGENT

(71) Applicants: THE UNIVERSITY OF TOKYO, Tokyo (JP); NIHON NOHYAKU CO., LTD., Tokyo (JP)

(72) Inventors: Kiyoshi KITA, Tokyo (JP); Akiyuki SUWA, Osaka (JP); Masatsugu ODA, Osaka (JP)

(73) Assignees: THE UNIVERSITY OF TOKYO, Tokyo (JP); NIHON NOHYAKU CO., LTD., Tokyo (JP)

(21) Appl. No.: 15/120,767

(22) PCT Filed: Mar. 4, 2015
(86) PCT No.: PCT/JP2015/056327

§ 371 Date: Aug. 23, 2016

(30) Foreign Application Priority Data
Mar. 5, 2014 (JP) 2014-042878

(51) Int. Cl. C07D 401/12; C07D 417/12; C07D 231/14; C07D 241/24; C07D 213/82; C07D 403/12; C07D 413/12

(52) U.S. Cl. CPC C07D 401/12; C07D 403/12; C07D 417/12; C07D 413/12; C07D 241/24; C07D 213/82; C07D 231/14

(57) ABSTRACT

The present invention is intended to provide a novel parasiticide, antiprotozoal or other endoparasite control agents which are effective for controlling animal endoparasites that have been impossible to control by conventional ones. Provided is an endoparasite control agent comprising, as an active ingredient, a heterocyclic carboxamide derivative represented by the general formula (I):

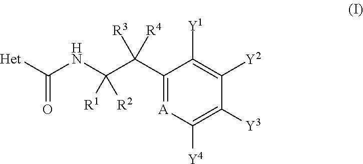

or a salt thereof.

### TECHNICAL FIELD

The present invention relates an endoparasite control agent comprising a heterocyclic carboxamide derivative or a salt thereof as an active ingredient, and a method for controlling endoparasites, comprising orally or parenterally administering the endoparasite control agent.

### BACKGROUND ART

Certain kinds of carboxamide derivatives have been known to have microbicidal activity (see Patent Literature 1 to 13). However, the literature does not describe that these compounds are affective for the disinfection or control of endoparasites in animals such as mammals and birds. It is also known that certain kinds of carboxamide derivatives are effective against nematodes that may damage agricultural products (see Patent Literature 4 or 5), but there is no specific disclosure as to whether these compounds are effective against endoparasites in animals, furthermore, there is a report that compounds that inhibit succinate-ubiquinone reductase (mitochondrial complex II), which is one of the respiratory enzymes of endoparasites, can serve as an endoparasite control agent (see Patent Literature 13 and Non Patent Literature 1). However, there is no disclosure of the compounds of the present invention.

Generally, parasitosis is caused, by infestation of host animals with parasites such as unicellular protists (protozoa), multicellular helminths and arthropods. It is reported that the incidence of parasitosis in Japan has been remarkably decreased by improvement of environmental hygiene, but on a global scale, particularly in -developing countries, parasitosis still widely prevails and causes tremendous damage. In recent years, there have been seen the introduction of infection sources: via long- or short-term travelers having visited such countries; parasitic infection due to the consumption of food imports or raw meat and fish meat, which have become more available thanks to the advance in freezing and logistics technologies; and the transmission of parasitosis from pets. Under such circumstances, the incidence of parasitosis is on an upward trend again. Another problem is that immunodeficiency caused by mass administration of immunosuppressants, anticancer drugs, etc. or by AIDS etc. allows usually non-pathogenic or low-pathogenic parasites to express their pathogenicity and so cause opportunistic infection in hosts. Further, parasitosis in domestic animals, such as pigs, horses, cattle, sheep, dogs, cats and domestic fowls, is a universal and serious economic problem. That is, parasitic infection of domestic animals causes anemia, malnutrition, debility, weight loss, and serious damage of intestinal tract walls, tissues and organs, and may result in decline in feed efficiency and productivity, leading to a great economic loss. Therefore, novel parasiticides, antiprotozoals or other endoparasite control, agents have always been desired.

### CITATION LIST

### Patent Literature

Patent Literature 1: JP-A 01-151546

Patent Literature 2: WO 2007/060162

Patent Literature 3: JP-A 53-9739

Patent Literature 4: WO 2007/108483

Patent Literature 5: WO 2007/104496

Parent Literature 6: WO 2008/101975

Patent literature 7: WO 2008/101976

Patent literature 8: WO 2008/003745

Patent literature 9: WO 2008/003746

Patent literature 10: WO 2009/012998

Patent literature 11: WO 2009/127718

Patent literature 12: WO 2010/106971

Patent literature 13: WO 2012/118139

### Non Patent Literature

Non Patent Literature 1: Kiyoshi Kita, “Kansen (Infection)” Winter 2010, Vol. 40-4, 310-319

### SUMMARY OF INVENTION

### Technical Problem

In view of the above-described circumstances, the present invention is mainly intended to provide a novel, parasiticide, antiprotozoal or other endoparasite control agents which are effective for controlling animal endoparasites that have been impossible to control by conventional ones.

### Solution to Problem

The present inventors conducted extensive research to solve the above-described problems. As a result, the present inventors found that a heterocyclic carboxamide derivative represented by the general formula (I) of the present invention and a salt thereof are highly effective for controlling endoparasites. The present inventors further conducted a great deal, of examination and then completed the present invention. That is, the present invention relates to the following.

[1] An endoparasite control agent comprising, as an active ingredient, a heterocyclic carboxamide derivative represented by the general formula (I):

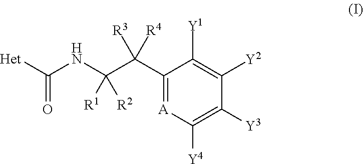

{wherein

R¹ and R² may be the same or different, and are selected from the group consisting of

(a1) a hydrogen atom;
(a2) a (C₁-C₆) alkyl group; and
(a3) a (C₁-C₆) alkoxy group, or optionally
(a4) R¹ and R² together with the carbon atom bound to R¹ and R² from a (C₃-C₄) cycloalkane,

R³ and R⁴ may be the same or different, and are selected from the group consisting of

(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₃-C₆) cycloalkane,

Y¹ is

(c1) a hydrogen atom;
(c2) a halogen atom;
(c3) a cyano group;
(c4) a nitro group;
(c5) a (C₁-C₆) alkyl group;
(c6) a (C₁-C₆) alkoxy group;
(c7) a halo (C₁-C₆) alkyl group; or
(c8) a halo (C₁-C₆) alkoxy group,

Y² and Y⁴ may be the same or different, and are selected from the group consisting of

(d1) a hydrogen atom;
(d2) a halogen atom;
(d3) a (C₁-C₆) alkyl group;
(d4) a (C₁-C₆) alkoxy group;
(d5) a halo (C₁-C₆) alkyl group; and
(d6) a halo (C₁-C₆) alkoxy group,

Y³ is selected from the group consisting of

(e1) a phenyl group;
(e2) a phenyl group having, on the ring, 1 to 5 substituting groups which may be the same or different and are selected from, the group consisting of (a) a halogen atom, (b) a cyano group, (c) a nitro group, (d) a (C₁-C₆) alkyl group, (a) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro: group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e6) a pyridyl group having, on the ring, 1 to 4 substituting groups which may be the same or different and are selected from the group: consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e7) a pyridyloxy group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e9) a pyrimidyloxy group;
(e10) a pyrimidyloxy group having, on the ring, 1 to 3 substituting groups which may be the same or different and are selected from the group consisting of (a) a halogen atom, (b) cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo a (C₁-C₆) alkyl group, (f) a (C₁-C₆) alkoxy group, (g) a halo (C₁-C₆) alkoxy group and (h) a formyl group;
(e15) a quinolyloxy group;
(e16) a quinolyloxy group having, on the ring, 1 to 6 substituting groups which may be the same or different and are selected from, the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e17) a quinoxalyloxy group;
(e18) a quinoxalyloxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e19) a benzoxazolyloxy group;
(e20) a benzoxazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e21) a benzothiazolyloxy group;
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e23) a furanyl group;
(e24) a furanyl group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e25) a thienyl group;
(e26) a thienyl group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e27) a naphthyl group;
(e28) a naphthyl group having, on the ring, 1 to 6 substituting groups which may be the same, or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e29) a naphthyloxy group; and
(e30) a naphthyloxy group having, on the ring, 1 to 6 substituting groups which may be the same or different and are selected, from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group,

A is a nitrogen atom or a CZ group (wherein Z represents a hydrogen atom, (a) a halogen atom, (d) a (C₁-C₆) alkyl group, (e) a halo (C₁-C₆) alkyl group, (f) a (C₁-C₆) alkoxy group or (g) a halo (C₁-C₆) alkoxy group), and

Het represents

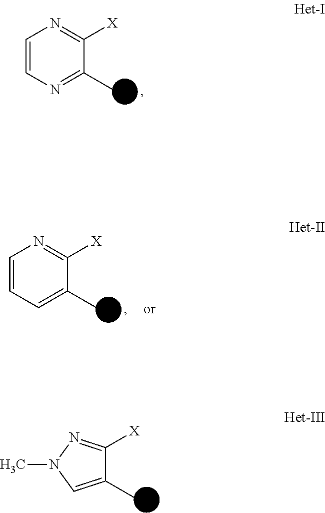

(wherein X represents a halogen atom or a halo (C₁-C₆) alkyl group, and the black circle represents a binding site)), or a salt thereof.
[2] The endoparasite control agent according to the above [1], wherein

R¹ and R² are (a1) hydrogen atoms,

R³ and R⁴ may be the same or different, and are selected from the group consisting of

(b1) a hydrogen atom;
(b2) a (C₁-C₆) alkyl group; and
(b3) a (C₁-C₆) alkoxy group, or optionally
(4) R³ and R⁴ together with the carbon atom bound to R³ and R⁴ form a (C₃-C₆) cycloalkane,

Y¹ is (c1) a halogen atom,

Y² and Y⁴ are (d1) hydrogen atoms, and

Y³ is selected from the group consisting of

(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (a) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e6) a pyridyl group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e7) a pyridyloxy group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e9) a pyrimidyloxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e16) a quinolyloxy group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e17) a quinoxalyloxy group;
(e18) a quinoxalyloxy group having, on the ring, 1 to 5 substituting groups which may be the same or different and are selected from, the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e19) a benzoxazolyloxy group;
(e20) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group.
[3] The endoparasite control agent according to the above [1], wherein

R¹ and R₂ are (a1) hydrogen atoms,

R³ and R⁴ may be the same or different, and are selected from the group consisting of

(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,

Y¹ is (c1) a halogen, atom,

Y² and Y⁴ are (d1) hydrogen atoms,

Y³ is selected from the group consisting of

(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a alkoxy group, (g) a halo (C₁-C₆) alkoxy group and (h) a formyl group;
(e15) a quinolyloxy group;
(e17) a quinoxalyloxy group;
(e19) a benzoxazolyloxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and

Het is Het-I,

[4] A method for controlling endoparasites, comprising orally or parenterally administering an effective amount of the endoparasite control agent according to any one of the above [1] to [3] to a non-human mammal or a bird.
[5] A method for controlling endoparasites, comprising orally or parenterally administering an effective amount of the endoparasite control agent according to any one of the above [1] to [3] to a non-human mammal.
[6] The method according to the above [4] or [5], wherein the non-human mammal is a domestic animal.
[7] A pyrazine carboxamide derivative represented by the general, formula (I):

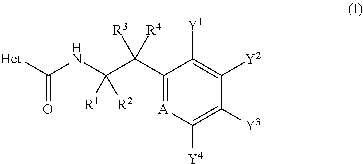

{wherein

R¹ and R² are (a1) hydrogen atoms,

R³ and R⁴ may be the same or different, and are selected from the group consisting of

(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,

Y¹ is (c1) a halogen atom,

Y² and Y⁴ are (d1) hydrogen atoms,

Y³ is selected from the group consisting of

(e1) a phenyl group;
(e2) a phenyl group having, on the ring, 1 to S substituting groups which may be the same or different and are selected from the group consisting of (a) a halogen atom, (b) a cyano group, (c) a nitro group, (d) a ((C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom., (to) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e17) a quinoxalyloxy group;
(e19) a benzoxazolyloxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, Co) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and

Het represents the following formula Het-I:

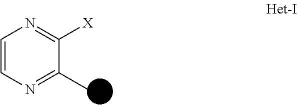

(wherein X represents a halogen atom or a halo (C₁-C₆) alkyl group, and the black circle represents a binding site)], or a salt thereof.
[8] A method for controlling endoparasites, comprising orally or parenterally administering an effective amount of the endoparasite control agent according to any one of the above [1] to [3] to a human.

[9] The heterocyclic carboxamide derivative specified in any one of the above [1] to [3] or a salt thereof for use in control of endoparasites.

[10] Use of the heterocyclic carboxamide derivative specified in any one of the above [1] to [3] or a salt thereof for production of endoparasite control agents.

[11] Use of the heterocyclic carboxamide derivative specified in any one of the above [1] to [3] or a salt thereof for control of endoparasites.

### ADVANTAGEOUS EFFECTS OF INVENTION

The present invention provides an endoparasite control agent having better performance in the disinfection or control of endoparasites as compared with the conventional art,

### DESCRIPTION OF EMBODIMENTS

The definitions in connection with the general formula (I)

representing the heterocyclic carboxamide derivative of the present invention are described below.

The “halogen atom” refers to a chlorine atom, a bromine atom, an iodine atom or a fluorine atom.

The “(C₁-C₆) alkyl group” refers to a straight-chain or branched-chain alkyl group of 1 to 6 carbon atoms, for example, a methyl group, an ethyl group, a n-propyl group, an isopropyl group, a n-butyl group, an isobutyl group, a sec-butyl group, a tert-butyl group, a n-pentyl group, a neopentyl group, a n-hexyl group or the like.

The “(C₁-C₆) alkoxy group” refers to a straight-chain or branched-chain alkoxy group of 1 to 6 carbon atoms, for example, a methoxy group, an ethoxy group, a n-propoxy group, an isopropoxy group, a n-butoxy group, a sec-butoxy group, a tert-butoxy group, a n-pentyloxy group, an isopentyloxy group, a neopentyloxy group, a n-hexyloxy group or the like.

The “halo (C₁-C₆) alkyl group” refers to a straight-chain or branched-chain alkyl group of 1 to 6carbon atoms substituted with one or more halogen atoms which may be the same or different from each other, for example, a trifluoromethyl group, a difluoromethyl group, a perfluoroethyl group, a hexafluoroisopropyl group, a perfluoroisopropyl group, a chloromethyl group, a bromomethyl group, a 1-bromoethyl group, a 2,3-dibromopropyl group or the like.

The “halo (C₁-C₆) alkoxy group” refers to a straight-chain or branched-chain alkoxy group of 1 to 6 carbon atoms substituted with one or more halogen atoms which may be the same or different from each other, for example, a trifluoromethoxy group, a difluoromethoxy group, a perfluoroethoxy group, a perfluoroisopropoxy group, a chloromethoxy group, a bromomethoxy group, a 1-bromoethoxy group, a 2,3-dibromopropoxy group or the like.

The “(C₁-C₆) cycloalkane” formed of R¹ and R² together with the carbon atom bound to R¹ and R² is, for example, cyclopropane, cyclobutane, cyclopentane, cyclohexane or the like,

Examples of the salt of the heterocyclic carboxamide derivative represented by the general formula (I) of the present invention include inorganic acid salts, such as hydrochlorides, sulfates, nitrates and phosphates; organic acid salts, such as acetates, fumarates, maleates, oxalates, methanesulfonates, benzenesulfonates and p-toluenesulfonates; and salts with an inorganic or organic base such as a sodium ion, a potassium ion, a calcium ion and a trimethylammonium ion.

As the heterocyclic carboxamide derivative of the present invention, preferred is a compound of the general formula (I) in which

R¹ and R² are (a1) hydrogen atoms,

R³ and R⁴ may be the same or different, and are selected from the group consisting of

(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,

Y¹ is (c1) a halogen atom,

Y² and Y⁴ are (d1) hydrogen atoms,

Y³ is selected from the group consisting of

(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a ((C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e6) a pyridyl group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e7) a pyridyloxy group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e9) a pyrimidyloxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e16) a quinolyloxy group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e17) a guinoxalykoxy group;
(e18) a quinoxalyloxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) a alkoxy group and (g) a halo (C₁-C₆) alkoxy group;
(e19) a benzoxazolyloxy group;
(e20) a benzoxazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and

Met is Het-I, Het-II or Het-III, or

a salt thereof.

As the heterocyclic carboxamide derivative of the present invention, further preferred is a compound of the general formula (I) in which

R¹and R² are (a1) hydrogen atoms,

R³ and R² may be the same or different, and are selected from the group consisting of

(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,

Y¹ is (c1) a halogen atom,

Y² and Y⁴ are (d1) hydrogen atoms,

Y³ is selected from the group consisting of

(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (c₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom., (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;

(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;

(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e17) a quinoxalyloxy group;
(e19) a benzoxazolyloxy group;
(e21) a benzothiazolyloxy group; and
(e22) a benzothiazolyloxy group having, on the ring, 1 to 4 substituting groups which may bhe same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and

Met is Het-I, Het-II or Het-I, or a salt thereof.

The compound represented by the general formula (I) of the present invention can be produced by the method described in JP-A 01-151546, WO 2007/060162, JP-A 53-9739, WO 2007/108483, WO 2008/101975, WO 2008/101976, WO 2008/003745, WO 2008/003746, WO 2009/012998, WO 2009/127718 or WO 2010/106971, the method described in WO 2012/118139 or Shin-Jikken Kagaku Kouza 14 (Maruzen, Dec. 20, 1977), a modified method of the foregoing, or the like.

Representative examples or the heterocyclic carboxamide derivative represented by the general formula (I) of the present invention are shown in Tables 1, 2 and 3, but the present invention is not limited thereto. In Tables 1, 3 and 3, “Me” stands for a methyl group, “Ph” stands for a phenyl group, “Py” stands for a pyridyl group, “PhO” stands for a phenoxy group, and “PyO” stands for a pyridyloxy group. Shown in the column of “Physical property” are mass spectral data.

Q1 to Q19 represent the following structures. The black circle in each of the formulae Q1 to Q19 represents a binding site.

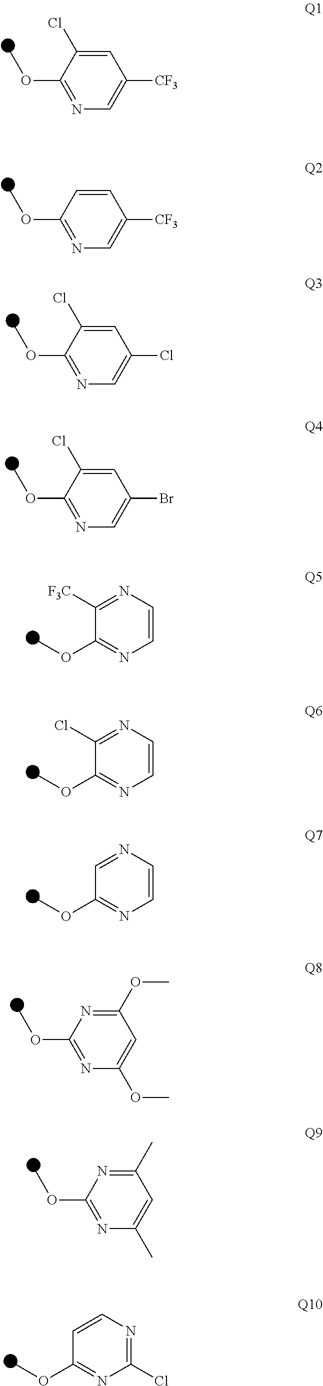

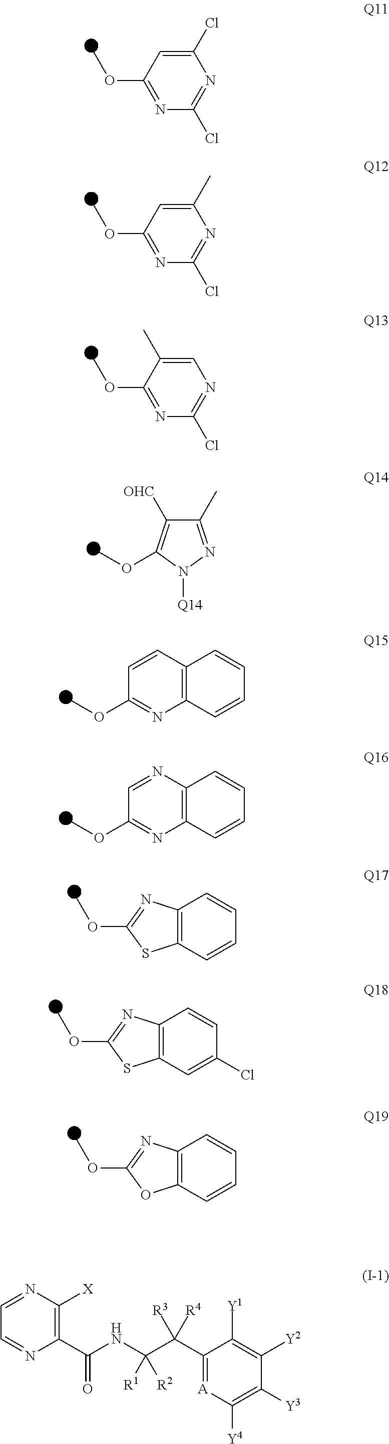

TABLE 1

| Compound | | | | | | Physical |
|---|---|---|---|---|---|---|
| No. | X | R³ | R⁴ | Y³ | A | property |
| 1-1 | CF₃ | H | H | Q1 | CH | 535 (M + 1) |
| 1-2 | CF₃ | H | H | Q2 | CH | 491 (M + 1) |
| 1-3 | CF₃ | H | H | Q3 | CH | 491 (M + 1) |
| 1-4 | CF₃ | H | H | Q4 | CH | 537 (M + 1) |
| 1-5 | CF₃ | H | H | Q5 | CH | 592 (M + 1) |
| 1-6 | CF₃ | H | H | Q6 | CH | 558 (M + 1) |
| 1-7 | CF₃ | H | H | Q7 | CH | 424 (M + 1) |
| 1-8 | CF₃ | H | H | Q8 | CH | 484 (M + 1) |
| 1-9 | CF₃ | H | H | Q9 | CH | 452 (M + 1) |
| 1-10 | CF₃ | H | H | Q10 | CH | 458 (M + 1) |
| 1-11 | CF₃ | H | H | Q11 | CH | 492 (M + 1) |
| 1-12 | CF₃ | H | H | Q12 | CH | 472 (M + 1) |
| 1-13 | CF₃ | H | H | Q13 | CH | 472 (M + 1) |
| 1-14 | CF₃ | H | H | Q14 | CH | 468 (M + 1) |
| 1-15 | CF₃ | H | H | Q15 | CH | 473 (M + 1) |
| 1-16 | CF₃ | H | H | Q16 | CH | 474 (M + 1) |
| 1-17 | CF₃ | H | H | Q17 | CH | 479 (M + 1) |
| 1-18 | CF₃ | H | H | Q18 | CH | 513 (M + 1) |
| 1-19 | CF₃ | H | H | Q19 | CH | 463 (M + 1) |
| 1-20 | CF₃ | H | H | PhO | CH | 422 (M + 1) |
| 1-21 | CF₃ | H | H | 4-CF₃—PhO | CH | 490 (M + 1) |
| 1-22 | CF₃ | Me | H | Q1 | CH | 539 (M + 1) |
| 1-23 | CF₃ | Me | H | Q2 | CH | 505 (M + 1) |
| 1-24 | CF₃ | Me | H | Q3 | CH | 505 (M + 1) |
| 1-25 | CF₃ | Me | Me | Q1 | CH | 553 (M + 1) |
| 1-26 | CF₃ | Me | Me | Q2 | CH | 519 (M + 1) |
| 1-27 | CF₃ | Me | Me | Q3 | CH | 519 (M + 1) |
| 1-28 | CF₃ | CH₂CH₂ | | Q1 | CH | 551 (M + 1) |
| 1-29 | CF₃ | CH₂CH₂ | | Q2 | CH | 517 (M + 1) |
| 1-30 | CF₃ | CH₂CH₂ | | Q3 | CH | 517 (M + 1) |
| 1-31 | CF₃ | H | H | 4-CF₃—Ph | CH | 474 (M + 1) |
| 1-32 | CF₃ | H | H | 3-CF₃—Ph | CH | 474 (M + 1) |
| 1-33 | CF₃ | H | H | 3,5-F₂—Ph | CH | 442 (M + 1) |
| 1-34 | CF₃ | H | H | 4-MeO—PhO | CH | 436 (M + 1) |
| 1-35 | CF₃ | H | H | Ph | CH | 406 (M + 1) |
| 1-36 | CF₃ | H | H | 4-Py | CH | 407 (M + 1) |
| 1-37 | CF₃ | H | H | 3-Py | CH | 407 (M + 1) |
| 1-38 | CF₃ | MeO | H | Q1 | CH | 555 (M + 1) |
| 1-39 | CF₃ | MeO | H | Q2 | CH | 521 (M + 1) |
| 1-40 | CF₃ | MeO | H | Q3 | CH | 523 (M + 1) |
| 1-41 | CF₃ | MeO | H | Q17 | CH | 509 (M + 1) |
| 1-42 | CF₃ | MeO | H | Ph | CH | 420 (—MeOH) |
| 1-43 | CF₃ | MeO | H | 4-CF₃—Ph | CH | 488 (—MeOH) |
| 1-44 | CF₃ | MeO | H | 4-MeO—PhO | CH | 450 (—MeOH) |
| 1-45 | CF₃ | MeO | H | 3-PyO | CH | 453 (—MeOH) |
| 1-46 | CF₃ | H | H | Ph | N | |
| 1-47 | CF₃ | H | H | 4-CF₃—Ph | N | |
| 1-48 | CF₃ | H | H | 3-CF₃—Ph | N | |
| 1-49 | CF₃ | H | H | 3,5-F₂—Ph | N | |
| 1-50 | CF₃ | Me | H | Ph | N | 421 (M + 1) |
| 1-51 | CF₃ | Me | H | 4-CF₃—Ph | N | |
| 1-52 | CF₃ | Me | H | 3-CF₃—Ph | N | |
| 1-53 | CF₃ | Me | H | 3,5-F₂—Ph | N | |
| 1-54 | CF₃ | Me | Me | Ph | N | |
| 1-55 | CF₃ | Me | Me | 4-CF₃—Ph | N | |
| 1-56 | CF₃ | Me | Me | 3-CF₃—Ph | N | |
| 1-57 | CF₃ | Me | Me | 3,5-F₂—Ph | N | |

In Table 1, R¹ and R² represent H, Y¹ represents Cl, and, Y² and Y⁴ represent H.

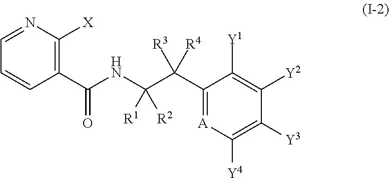

TABLE 2

| Compound | | | | | | Physical |
|---|---|---|---|---|---|---|
| No. | X | R³ | R⁴ | Y³ | A | property |
| 2-1 | CF₃ | H | H | Q1 | CH | |
| 2-2 | CF₃ | H | H | Q2 | CH | |
| 2-3 | CF₃ | H | H | Q3 | CH | |
| 2-4 | CF₃ | H | H | Q4 | CH | |
| 2-5 | CF₃ | H | H | Q1 | CH | |
| 2-6 | CF₃ | H | H | Q2 | CH | |
| 2-7 | CF₃ | H | H | Q3 | CH | |
| 2-8 | CF₃ | H | H | Q4 | CH | |
| 2-9 | CF₃ | H | H | Q1 | CH | |
| 2-10 | CF₃ | H | H | Q2 | CH | |
| 2-11 | CF₃ | H | H | Q3 | CH | |
| 2-12 | CF₃ | H | H | Q4 | CH | |
| 2-13 | CF₃ | H | H | Q1 | CH | |
| 2-14 | CF₃ | H | H | Q2 | CH | |
| 2-15 | CF₃ | H | H | Q3 | CH | |
| 2-16 | CF₃ | H | H | Q4 | CH | |
| 2-17 | CF₃ | H | H | Q1 | CH | |
| 2-18 | CF₃ | H | H | Q2 | CH | |
| 2-19 | CF₃ | H | H | Q3 | CH | |
| 2-20 | CF₃ | H | H | PhO | CH | |
| 2-21 | CF₃ | H | H | 4-CF₃—PhO | CH | |
| 2-22 | CF₃ | Me | H | Q1 | CH | |
| 2-23 | CF₃ | Me | H | Q2 | CH | |
| 2-24 | CF₃ | Me | H | Q3 | CH | |
| 2-25 | CF₃ | Me | Me | Q1 | CH | |
| 2-26 | CF₃ | Me | Me | Q2 | CH | |
| 2-27 | CF₃ | Me | Me | Q3 | CH | |
| 2-28 | CF₃ | CH₂CH₂ | | Q1 | CH | |
| 2-29 | CF₃ | CH₂CH₂ | | Q2 | CH | |
| 2-30 | CF₃ | CH₂CH₂ | | Q3 | CH | |
| 2-31 | CF₃ | H | H | 4-CF₃—Ph | CH | |
| 2-32 | CF₃ | H | H | 3-CF₃—Ph | CH | |
| 2-33 | CF₃ | H | H | 3,5-F₂—Ph | CH | |
| 2-34 | CF₃ | H | H | 4-MeO—PhO | CH | |
| 2-35 | CF₃ | H | H | Ph | CH | |
| 2-36 | CF₃ | H | H | 4-Py | CH | |
| 2-37 | CF₃ | H | H | 3-Py | CH | |
| 2-38 | CF₃ | MeO | H | Q1 | CH | |
| 2-39 | CF₃ | MeO | H | Q2 | CH | |
| 2-40 | CF₃ | MeO | H | Q3 | CH | |
| 2-41 | CF₃ | MeO | H | Q17 | CH | |
| 2-42 | CF₃ | MeO | H | PhO | CH | |
| 2-43 | CF₃ | MeO | H | 4-CF₃—PhO | CH | |
| 2-44 | CF₃ | MeO | H | 4-MeO—PhO | CH | |
| 2-45 | CF₃ | MeO | H | 3-PyO | CH | |
| 2-46 | Cl | H | H | Ph | N | |
| 2-47 | Cl | H | H | 4-CF₃—Ph | N | |
| 2-48 | Cl | H | H | 3-CF₃—Ph | N | |
| 2-49 | Cl | H | H | 3,5-F₂—Ph | N | |
| 2-50 | Cl | Me | H | Ph | N | |
| 2-51 | Cl | Me | H | 4-CF₃—Ph | N | |
| 2-52 | Cl | Me | H | 3-CF₃—Ph | N | |
| 2-53 | Cl | Me | H | 3,5-F₂—Ph | H | |
| 2-54 | Cl | Me | Me | Ph | N | |
| 2-55 | Cl | Me | Me | 4-CF₃—Ph | N | |
| 2-56 | Cl | Me | Me | 3-CF₃—Ph | N | |
| 2-57 | Cl | Me | Me | 3,5-F₂—Ph | N | |

In Table 2, R¹ and R² represent H, Y¹ represents Cl, and Y² and Y⁴ represent H.

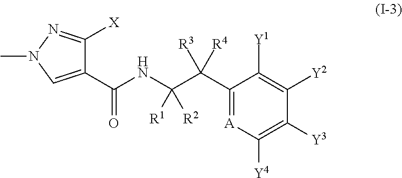

TABLE 3

| Compound | | | | | | Physical |
|---|---|---|---|---|---|---|
| No. | X | R³ | R⁴ | Y³ | A | property |
| 3-1 | CF₃ | H | H | Q1 | CH | |
| 3-2 | CF₃ | H | H | Q2 | CH | |
| 3-3 | CF₃ | H | H | Q3 | CH | |
| 3-4 | CF₃ | H | H | Q4 | CH | |
| 3-5 | CF₃ | H | H | Q5 | CH | |
| 3-6 | CF₃ | H | H | Q6 | CH | |
| 3-7 | CF₃ | H | H | Q7 | CH | |
| 3-8 | CF₃ | H | H | Q8 | CH | |
| 3-9 | CF₃ | H | H | Q9 | CH | |
| 3-10 | CF₃ | H | H | Q10 | CH | |
| 3-11 | CF₃ | H | H | Q11 | CH | |
| 3-12 | CF₃ | H | H | Q12 | CH | |
| 3-13 | CF₃ | H | H | Q13 | CH | |
| 3-14 | CF₃ | H | H | Q14 | CH | |
| 3-15 | CF₃ | H | H | Q15 | CH | |
| 3-16 | CF₃ | H | H | Q16 | CH | |
| 3-17 | CF₃ | H | H | Q17 | CH | |
| 3-18 | CF₃ | H | H | Q18 | CH | |
| 3-19 | CF₃ | H | H | Q19 | CH | |
| 3-20 | CF₃ | H | H | PhO | CH | |
| 3-21 | CF₃ | H | H | 4-CF₃—Ph | CH | |
| 3-22 | CF₃ | Me | H | Q1 | CH | |
| 3-23 | CF₃ | Me | H | Q2 | CH | |
| 3-24 | CF₃ | Me | H | Q3 | CH | |
| 3-25 | CF₃ | Me | Me | Q1 | CH | |
| 3-26 | CF₃ | Me | Me | Q2 | CH | |
| 3-27 | CF₃ | Me | Me | Q3 | CH | |
| 3-28 | CF₃ | CH₂CH₂ | | Q1 | CH | |
| 3-29 | CF₃ | CH₂CH₂ | | Q2 | CH | |
| 3-30 | CF₃ | CH₂CH₂ | | Q3 | CH | |
| 3-31 | CF₃ | H | H | 4-CF₃—Ph | CH | |
| 3-32 | CF₃ | H | H | 3-CF₃—Ph | CH | |
| 3-33 | CF₃ | H | H | 3,5-F₂—Ph | CH | |
| 3-34 | CF₃ | H | H | 4-MeO—PhO | CH | |
| 3-35 | CF₃ | H | H | Ph | CH | |
| 3-36 | CF₃ | H | H | 4-Py | CH | |
| 3-37 | CF₃ | H | H | 3-Py | CH | |
| 3-38 | CF₃ | MeO | H | Q1 | CH | |
| 3-39 | CF₃ | MeO | H | Q2 | CH | |
| 3-40 | CF₃ | MeO | H | Q3 | CH | |
| 3-41 | CF₃ | MeO | H | Q17 | CH | |
| 3-42 | CF₃ | MeO | H | PhO | CH | |
| 3-43 | CF₃ | MeO | H | 4-CF₃—PhO | CH | |
| 3-44 | CF₃ | MeO | H | 4-MeO—PhO | CH | |
| 3-45 | CF₃ | MeO | H | 3-PyO | CH | |
| 3-46 | CHF₂ | H | H | Q1 | CH | |
| 3-47 | CHF₂ | H | H | Q2 | CH | |
| 3-48 | CHF₂ | H | H | Q3 | CH | |
| 3-49 | CHF₂ | H | H | Q4 | CH | |
| 3-50 | CHF₂ | H | H | Q5 | CH | |
| 3-51 | CHF₂ | H | H | Q6 | CH | |
| 3-52 | CHF₂ | H | H | Q7 | CH | |
| 3-53 | CHF₂ | H | H | Q8 | CH | |
| 3-54 | CHF₂ | H | H | Q9 | CH | |
| 3-55 | CHF₂ | H | H | Q10 | CH | |
| 3-56 | CHF₂ | H | H | Q11 | CH | |
| 3-57 | CHF₂ | H | H | Q12 | CH | |
| 3-58 | CHF₂ | H | H | Q13 | CH | |
| 3-59 | CHF₂ | H | H | Q14 | CH | |
| 3-60 | CHF₂ | H | H | Q15 | CH | |
| 3-61 | CHF₂ | H | H | Q16 | CH | |
| 3-62 | CHF₂ | H | H | Q17 | CH | |
| 3-63 | CHF₂ | H | H | Q18 | CH | |
| 3-64 | CHF₂ | H | H | Q19 | CH | |
| 3-65 | CHF₂ | H | H | PhO | CH | |
| 3-66 | CHF₂ | H | H | 4-CF₃—PhO | CH | |
| 3-67 | CHF₂ | Me | H | Q1 | CH | |
| 3-68 | CHF₂ | Me | H | Q2 | CH | |
| 3-69 | CHF₂ | Me | H | Q3 | CH | |
| 3-70 | CHF₂ | Me | Me | Q1 | CH | |
| 3-71 | CHF₂ | Me | Me | Q2 | CH | |
| 3-72 | CHF₂ | Me | Me | Q3 | CH | |
| 3-73 | CHF₂ | CH₂CH₂ | | Q1 | CH | |
| 3-74 | CHF₂ | CH₂CH₂ | | Q2 | CH | |
| 3-75 | CHF₂ | CH₂CH₂ | | Q3 | CH | |
| 3-76 | CHF₂ | H | H | 4-CF₃—Ph | CH | |
| 3-77 | CHF₂ | H | H | 3-CF₃—Ph | CH | |
| 3-78 | CHF₂ | H | H | 3,5-F₂—Ph | CH | |
| 3-79 | CHF₂ | H | H | 4-MeO—PhO | CH | |
| 3-80 | CHF₂ | H | H | Ph | CH | |
| 3-81 | CHF₂ | H | H | 4-Py | CH | |
| 3-82 | CHF₂ | H | H | 3-Py | CH | |
| 3-83 | CHF₂ | MeO | H | Q1 | CH | |
| 3-84 | CHF₂ | MeO | H | Q2 | CH | |
| 3-85 | CHF₂ | MeO | H | Q3 | CH | |
| 3-86 | CHF₂ | MeO | H | Q17 | CH | |
| 3-87 | CHF₂ | MeO | H | PhO | CH | |
| 3-88 | CHF₂ | MeO | H | 4-CF₃—PhO | CH | |
| 3-89 | CHF₂ | MeO | H | 4-MeO—PhO | CH | |
| 3-90 | CHF₂ | MeO | H | 3-PyO | CH | |
| 3-91 | CF₃ | H | H | Ph | N | |
| 3-92 | CF₃ | H | H | 4-CF₃—Ph | N | |
| 3-93 | CF₃ | H | H | 3-CF₃—Ph | N | |
| 3-94 | CF₃ | H | H | 3,5-F₂—Ph | N | |
| 3-95 | CF₃ | Me | H | Ph | N | |
| 3-96 | CF₃ | Me | H | 4-CF₃Ph | N | |
| 3-97 | CF₃ | Me | H | 3-CF₃—Ph | N | |
| 3-98 | CF₃ | Me | H | 3,5-F₂—Ph | N | |
| 3-99 | CF₃ | Me | Me | Ph | N | |
| 3-100 | CF₃ | Me | Me | 4-CF₃—Ph | N | |
| 3-101 | CF₃ | Me | Me | 3-CF₃—Ph | N | |
| 3-102 | CF₃ | Me | Me | 3,5-F₂—Ph | N | |
| 3-103 | CHF₂ | H | H | Ph | N | |
| 3-104 | CHF₂ | H | H | 4-CF₃—Ph | N | |
| 3-105 | CHF₂ | H | H | 3-CF₃—Ph | N | |
| 3-106 | CHF₂ | H | H | 3,5-F₂—Ph | N | |
| 3-107 | CHF₂ | Me | H | Ph | N | |
| 3-108 | CHF₂ | Me | H | 4-CF₃—Ph | N | |
| 3-109 | CHF₂ | Me | H | 3-CF₃—Ph | N | |
| 3-110 | CHF₂ | Me | H | 3,5-F₂—Ph | N | |
| 3-111 | CHF₂ | Me | Me | Ph | N | |
| 3-112 | CHF₂ | Me | Me | 4-CF₃—Ph | N | |
| 3-113 | CHF₂ | Me | Me | 3-CF₃—Ph | N | |
| 3-114 | CHF₂ | Me | Me | 3,5-F₂—Ph | N | |

In Table 3, R¹ and R² represent H, Y¹ represents Cl, and Y² and Y⁴ represent H.

The endoparasite control agent of the present invention has excellent anti-endoparasite effect, and exerts appropriate control effect against endoparasites. The animal for which the endoparasite control agent of the present invention can be used is a human and an animal of non-human mammalian or avian species. Exemplary members of the non-human mammalian species include domestic animals, such as pigs, horses, cattle, sheep, goats, rabbits, camels, water buffalos, deer, mink and chinchillas; pet animals, such as dogs, cats, little birds and monkeys; and experimental animals, such as rats, mice, golden hamsters and guinea pigs. Exemplary members of the avian species include domestic fowls, such as chickens, ducks, aigamo ducks (crossbreeds of wild and domestic ducks), quails, domestic ducks, geese and turkeys. The examples listed above are non-limiting examples.

Human endoparasites against which the endoparasite control agent of the present invention is effective are roughly classified into protozoa and helminths. Examples of the protozoa include, but are not limited thereto, Rhizopoda, such as Entamoeba histolytica; Mastigophora, such as Leishmania, Trypanosoma and Trichomonas; Sporozoea, such as Plasmodium and Toxoplasma; and Ciliophora, such as Balantidium coli. Examples of the helminths include, but are not limited thereto, Nematoda, such as Ascaris lumbricoides, Anisakis, Toxocara canis, Trichostrongylus spp., Enterobius vermicularis, hookworms (for example, Ancylostoma duodenale, Nector americanus, Ancylostoma braziliense, etc.), Angiostrongylus spp., Gnathostoma spp., filarial worms (filaria, Wuchereria bancrofti, Bragia malayi, etc.), Onchocerca volvulus, Dracunculus medinensis, Trichinella spiralis and Strongyloides stercoralis; Acanthocephala, such as Macracanthorhynchus hirudinaceus; Gordiacea, such as Gordioidea; Hirudinea, such as Hirudo nipponia; Trematoda, such as Schistosoma japonicum, Schistosoma mansoni, Schistosoma haematobium, Clonorchis sinensis, Heterophyes heterophyes, Fasciola spp. and Paragonimus spp.; and Cestoda, such as Diphyllobothrium latum Sparganum mansoni, Sparganum proliferum, Diplogonoporus grandis, Taeniidae (for example, Taeniarhynchus saginatus, Taenia solium, Echinococcus, etc.), Hymenolepis spp., Dipylidium caninum, Mesocestoides lineatus, Bertiella spp.. and Nybelinia surmenicola.

Non-human mammalian or avian endoparasites against which the endoparasite control agent of the present invention is effective are roughly classified into protozoa and helminths. Examples of the protozoa include, but are not limited thereto, Apicomplexa, such as Coccidia (for example, Eimeria, Isospora, Toxoplasma, Neospora, Sarcocystis, Besnoitia, Hammondia, Cryptosporidium, Caryospora, etc.), Haemosporina (for example, Leucocytozoon, Plasmodium, etc.), Piroplasma (for example, Theileria, Anaplasma, Eperythrozoon Haemobartonella, Ehrlichia, etc.), and others (for example, Hepatozoon, Haemogregarina, etc.); Microspore, such as Encephalitozoon and Nosema; Mastigophora, such as Trypanosomatid (for example, Trypanosoma, leishmania, etc.), Trichomonadida (for example, Chilomastix, Trichomonas, Monocercomonas, Histomonas, etc.), and Diplomonadida (for example, Hexamita, Giardia, etc.); Sarcodina, such as Amoebida (for example, Entamoeba histolytica (Entamoeba) etc.); and Ciliophora, such as Balantidium coli (Balantidium), Buxtonella and Entodinium.

Examples of the helminths include, but are not limited thereto, Nematoda, such as Ascaridida (for example, Ascaris suum (Ascaris), Toxocara canis and Toxocara cati (Toxocara), Toxascaris leonina (Toxascaris), Parascaris equorum (Parascaris), Ascaridia galli (Ascaridia), Hetarakis gallinarum (Heterakis), Anisakis, etc.), Oxyurida (for example, Oxyuris equi (Oxyuris), Passalurus ambiguus (Passalurus), etc.), Strongylida (for example, Strongylus vulgaris (Strongylus), Haemonchus contortus (Haemonchus), Ostertagia ostertagi (Ostertagia), Trichostrongylus colubriformia (Trichostrongylus), Cooperia punctata (Cooperia), Nematodirus filicollis (Nematodirus), Hyostrongylus rubidus (Hyostrongylus), Oesophagostomun radiatum (Oesophagostomum), Chabertia ovina (Chabertia), Ancylostoma caninum (Ancylostoma), Uncinaria stenocephala (Uncinaria), Necator americanus (Necator), Bunostomum phlebotomum (Bunostomum), Dictyocaulus viviparus (Dictyocaulus), Metastrongylus elongatus (Metastrongylus), Filaroides hirthi (Filaroides), Aelurostrongylus abstrusus (Aelurostrongylus), Angiostrongylus cantonensis (Angiostrongylus), Syngamus trachea (Syngamus), Stephanurus dentatus (Stephanurus), etc.), Rhabditida (for example, Strongyloses stercoralis (Strongyloides (Micronema, etc.), Spirurida (for example, Thelazia rhodesi (Thelazia), Oxyspirura mansoni (Oxyspirura), Spirocerca lupi (Spirocerca), Gongylonema pulchrum (Gongylonema), Draschia megastoma (Draschia), Habronema microstoma (Habronema), Ascarops strongylina (Ascarops), Physaloptera praeputialis (Physaloptera), Gnathostoma spinigerum (Gnathostoma), etc.), Filariida (for example, Dirofilaria immitis (Dirofilaria), Setaria equina (Setaria), Dipetalonema, Parafilaria multipapillosa (Parafilaria), Onchocerca cervicalis (Onchocerca), etc.), and Enoplida (for example, Parafilaria bovicola (Parafilaria), Stephanofilaria okinawaensis (Stephanofilaria), Trichuris vulpis (Trichuris), Capillaria bovis (Capillaria), Trichosomoides crassicauda (Trichosotnoides), Trichinella spiralis (Trichinella), Dioctophyma renale (Dioctophyma), etc.); Trematoda, such as Fasciolata (for example, Fasciola hepatica (Fasciala), Fasciolopsis buski (Fasciolopsis), etc.), Paramphistomatidae (for example, Homalogaster paloniae (Homalogaster), etc.), Dicrocoelata (for example, Eurytrema pancreaticum (Eurytrema), Dicrocoelium dendriticum (Dicrocoelium), etc.), Diplostomata (for example, Pharyngostomum cordatum (Pharyngostomum), Alaria, etc.), Echinostomata (for example, Echinostoma hortense (Echinostoma), Echinochasmus, etc.), Troglotrematoidea (for example, lung flukes (Paragonimus), Nanophyetus salmincola (Nanophyetus), etc.), Opisthorchiida (for example, Clonorchis sinensis (Clonorchis) etc.), Heterophyida (for example, Heterophyes heterophyes (Heterophyes), Metagonimus yokogawai (Metagonimus), etc.), Plagiorchiida (for example, Prosthogonimus ovatus (Prosthogonimus) etc.), and Schistosomatidae (for example, Schistosoma japonicum (Schistosoma) etc.); Cestoda, such as Pseudophylidea (for example, Diphyllobothrium nihonkaiense (Diphyllobothrium), Spirometra erinacei (Spirometra), etc.), and Cyclophyllidea (for example, Anopiocephala perfoliata (Anoplocephala), Spirometra erinacei (Spirometra), etc.), (Paranoplocephala), Moniezia benedeni (Moniezia), Dipylidium caninum (Dipylidium), Mesocestoides lineatus (Mesocestoides), Taenia pisiformis and Taenia hydatigena (Taenia), Hydatigera taeniaeformis (Hydatigera), Multiceps multiceps (Multiceps), Echinococcus granulosus (Echinococcus), Echinococcus multilocularis (Echinococcus), Taenia solium (Taenia), Taeniarhynchus saginatus (Taeniarhynchus), Hymenolepis diminuta (Hymenolepis), Vampirolepis nana (Vampirolepis), Raillietina tetragons (Raillietina), Amoebotaenia sphenoides (Amoebotaenia), etc.); Acanthocephala, such as Macracanthorhynchus hirudinaceus (Macracanthorhynchus) and Moniliformis, moniliformis. (Moniliformis); Linguatulida, such as Linguatula serrata (Linguatula); and other various parasites.

In different designations, examples of the helminths include, but are not limited to, Nematoda, such as Enoplida (for example, Trichuris spp., Capillaria spp., Trichomosoides spp., Trichinella spp., etc.), Rhabditia (for example, Micronema spp., Strongyloides spp., etc.), Strongylida (for example, Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Sunostomum spp., Globocepnalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp. Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostonum spp., Ollulanus spp., etc.),

Oxyurida (for example, Qxyuris spp., Enterobius spp., Passalurus spp., Syphacia spp., Aspiculuris spp., Heterakis spp., etc.), Ascaridra (for example, Ascaris spp., Toxascaris spp., Toxocara spp., Parascaris spp., Anisakis spp., Ascaridia spp., etc.), Spirurida (for example, Gnathostoma spp., Physaloptera spp., Thelazia spp., Gongylonema spp., Habronema spp., Parabronema spp., Draschia spp., Dracunculus spp., etc.), and Filariida (for example, Stephanofilaria spp., Parafilaria spp., Setaria spp., Loa spp., Dirofilaria spp., Litomosoides spp., Brugia spp., Wuchereria spp., Onchocerca spp., etc.);

Acanthocephala (for example, Filicollis spp., Moniliformis spp., Macracanthorhynchus spp., Prosthenorchis spp., etc,); Trematoda including subclasses, such as Monogenea (for example, Gyrodactylus spp., Dactylogyrus spp., Polystoma spp., etc.) and Digenea (for example, Diplostomum spp., Posthodiplostomum spp., Schistosoma spp., Trichobilharzia spp., Ornithbilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantcotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Cionorchis spp., Metorchis spp., Heterophyes spp., Metagonimus spp., etc.);

Cestoda, such as Pseudophyllidea (for example, Diphyllobothrium spp., Spirometra spp., Schistocephalus spp., Ligula spp., Bothridium spp., Diplogonoporus spp., etc,), and Cyclophyllidea (for example, Mesocestoides spp., Anoplocephala spp., Paranoplocehala spp., Moniezia spp., Thysanosomsa spp., Thysaniezia spp., Avitellina spp., Stilesia spp., Cittotaenia spp., Andyra spp., Bertiella spp., Taenia spp., Echinococcws spp., Hydatigera spp., Davainea spp., Raillietina. spp., Hymenolepis spp., Echinolepis spp., Echinocotyle spp., Diorchis spp., Dipylidium spp., Joyeuxiella spp., Diplopylidium spp., etc.); and others including parasites belonging to Acanthocephala and Linguatulida.

The endoparasite control agent of the present invention is effective for controlling not only parasites that live in the body of an intermediate or final host, but also parasites that, live in the body of a reservoir host. The compound represented by the general formula (I) of the present invention is effective for controlling parasites at their every developmental stage. For example, in the case of protozoa, the compound is effective against their cysts, precystic forms and trophozoites; schizonts and amoeboid forms at the asexual stage; gametocytes, gametes and zygotes at the sexual stage; sporozoites; etc. In the case of nematodes, the compound, is effective against their eggs, larvae and adults. The compound of the present invention is capable of not only combating parasites in the living body, but also even preventing parasitic infection by application to the environment as a route of infection. For example, soli-borne infection, i.e., infection from soil of crop fields and parks; percutaneous infection from water in rivers, lakes, marshes, paddy fields, etc.; oral infection from feces of animals such as dogs and cats; oral infection, from saltwater fish, freshwater fish, crustaceans, shellfish, raw meat of domestic animals, etc.; infection from mosquitoes, gadflies, flies, cockroaches, mites and ticks, fleas, lice, assassin bugs, trombiculid mites, etc.; and the like can be prevented from occurring.

The endoparasite control agent of the present invention can be administered as a pharmaceutical for treatment or prevention of parasitosis in humans and animals of non-human mammalian or avian species. The mode of administration may be oral or parenteral administration. In the case of oral administration, the endoparasite control agent of the present invention can be administered, for example, as a capsule, a tablet, a pill, a powder, a granule, a fine granule, a powder, a syrup, an enteric-coated preparation, a suspension or a paste, or after blended in a liquid drink or feed for animals. In the case of parenteral administration, the endoparasite control agent of the present invention, can be administered, for example, as an injection, an infusion, a suppository, an emulsion, a suspension, a drop, an ointment, a cream, a solution, a lotion, a spray, an aerosol, a cataplasm or a tape, or in a dosage form which allows sustained mucosal or percutaneous absorption.

In the case where the endoparasite control agent of the present invention is used as a pharmaceutical for humans and animals of non-human mammalian or avian species, the optimum amount (effective amount) of the active ingredient varies with the purpose (treatment or prevention), the kind of infectious parasite, the type and severity of infection, the dosage form, etc., hut in general, the oral daily dose is in the range of about 0.0001 to 10000 mg/kg body weight and the parenteral daily dose is in the range of about 0.0001 to 10000 mg/kg body weight. Such a dose may be given as a single dose or divided into multiple doses.

The concentration of the active ingredient in the endoparasite control agent of the present invention is generally about 0.001 to 100% by mass, preferably about 0.001 to 99% by mass, and more preferably about 0.005 to 20% fey mass. The endoparasite control agent of the present invention may be a composition that can be directly administered, or a highly concentrated composition that needs to be diluted to a suitable concentration before use.

The endoparasite control agent of the present Invention can be used in combination with any existing endoparasite control agent for the purpose of reinforcing or complementing its effect. In such a combined use, two or more active ingredients may be mixed and formulated into a single preparation before administration, or two or more different preparations may be administered separately.

### EXAMPLES

Next, the present invention will toe illustrated in detail by formulation examples and test example of the endoparasite control agent of the present invention, but the scope of the present invention is not limited by the following formulation examples and test example.

In Examples, “part” means a part by mass,

### Formulation Example 1 (Emulsion)

Ten parts of a heterocyclic carboxamide derivative represented by the general formula (I) of the present invention, 6 parts of Sorpol 355S (surfactant, manufactured by Toho Chemical Industry), and 84 parts of Solvesso 150 (manufactured by Exxon) are uniformly mixed with stirring to give an emulsion.

### Formulation Example 2 (Ointment)

One part of a heterocyclic carboxamide derivative represented by the general, formula (I) of the present invention, 50 parts of white beeswax, and 49 parts of white petrolatum are well mixed to give an ointment,

### Formulation Example 3 (Tablet)

Two parts of a heterocyclic carboxamide derivative represented by the general formula (I) of the present invention, 10 parts of vegetable oil (olive oil), 3 parts of crystalline cellulose, 20 parts of white carbon, and 65 parts of kaolin are well mixed and compressed into a tablet.

### Formulation Example 4 (Injection)

Ten parts of a heterocyclic carboxamide derivative represented by the general formula (I) of the present invention, 10 parts of propylene glycol for use as a food additive,, and 80 parts of vegetable oil (corn oil) are mixed to give an injection.

### Formulation Example 5 (Solution)

Five parts of a heterocyclic carboxamide derivative represented by the general formula (I) of the present invention, 20 parts of a surfactant for ordinary use as a dissolution or suspension aid, and 75 parts of ion exchanged water are well mixed to give a solution,

### Test Example

Test for Effect on Motion of Larvae of Haemonchus Nematode (Haemonchus contortus)

The compound of the present invention was prepared as solutions in 100% DMSO at the final concentrations of 50 ppm, 5 ppm, 0.5 ppm, 0.05 ppm and 0.005 ppm. DMSO stands for dimethyl sulfoxide.

A larval suspension containing 1st-stage larvae of Haemonchus contortus harvested by the Baermann technique (for example, see K. Nakazono et al., “Inclination of Baermann funnel wall and efficiency of nematode extraction” Proc. Assoc. Pl. Prot. Kyushu 33; 126-130 (1987) was placed at a density of 20 larvae per well in a test place, and 0.5 μL/well of the test solution containing the compound of the present invention diluted to a predetermined concentration was added to the test plate. The plate was kept, under the conditions of 27° C./95% RH for 4 days. In the test, ivermectin was used for the positive control and DMSO was used for the negative control.

The motor ability of the larvae was examined with an automatic analyzer equipped with an LCD camera. The inhibitory effect on the motion of the larvae in each treatment plot was corrected based on the inhibitory effect in the plot treated with DMSO only for the negative control. The EC₅₀ value was calculated from the data on the corrected inhibitory effect on the motion of the larvae, and graded according to the criteria shown below.

The test was conducted in duplicate per plot.

Grading Criteria

EC₅₀ value:

| | |
|---|---|
| 0.05 ppm or less | A |
| 0.05 to 1 ppm | B |
| 1 to 10 ppm | C |
| 10 ppm or more | D |

As a result, the compounds 1-2, 1-3, 1-4, 1-5, 1-6, 1-7, 1-8, 1-10, 1-11, 1-12, 1-13, 1-14, 1-15, 1-16, 1-17, 1-18, 1-19, 1-21, 1-22, 1-23, 1-24, 1-25, 1-26, 1-27, 1-28, 1-29, 1-30, 1-31, 1-32, 1-33, 1-34, 1-35, 1-1-36, 1-37, 1-38, 1-39, 1-40, 1-41, 1-1-42 1-43, 1-44 and 1-45 of the present invention showed the activity level graded as C or higher. The results show that the compounds of the present invention are effective as an endoparasite control agent.

1-11. (canceled)

12. An endoparasite control agent comprising, as an active ingredient, a heterocyclic carboxamide derivative represented by the general formula (I): 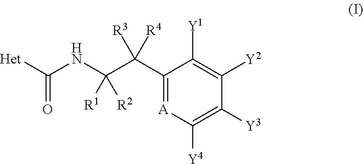
{wherein
R¹ and R² may be the same or different, and are selected from the group consisting of
(a1) a hydrogen atom;
(a2) a (C₁-C₆) alkyl group; and
(a3) a (C₁-C₆) alkoxy group, or optionally
(a4) R¹ and R² together with the carbon atom bound to R¹ and R² form a (C₁-C₆) cycloalkane,
R³ and R⁴ may be the same or different, and are selected from the group consisting of
(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,
Y¹ is
(c1) a hydrogen atom;
(c2) a halogen atom;
(c3) a cyano group;
(c4) a nitro group;
(c5) a (C₁-C₆) alkyl group;
(c6) a (C₁-C₆) alkoxy group;
(c7) a halo (C₁-C₆) alkyl group; or
(c8) a halo (C₁-C₆) alkoxy group,
Y² and Y⁴ may be the same or different, and are selected from the group consisting of
(d1) a hydrogen atom;
(d2) a halogen atom;
(d3) a (C₁-C₆) alkyl group;
(d4) a (C₁-C₆) alkoxy group;
(d5) a halo (C₁-C₆) alkyl group; and
(d6) a halo (C₁-C₆) alkoxy group,
Y³ is selected from the group consisting of
(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆), (f) a (C₁-C₆) alkoxy group and (g) a halo (C₁-C₆) alkoxy group;
(e5) a pyridyl group;
(e6) a pyridyl group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e7) a pyridyloxy group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e9) a pyrimidyloxy group;
(e10) a pyrimidyloxy group having, on the ring, 1 to 3 substituting groups which may be the same or different and are selected from the group consisting of (a) a halogen atom, (b) a cyano group, (e) a nitro group, (d) a (C₁-C₆) alkyl group, (e) a halo (C₁-C₆) alkyl group, (f) a (C₁-C₆) alkoxy group and (g) a halo (C₁-C₆) alkoxy group,
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e16) a quinolyloxy group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group,
(e17) a quinoxalyloxy group;
(e18) a quinoxalyloxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e19) a benzoxazolyloxy group;
(e20) a benzoxazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e21) a benzothiazolyoxy group; and
(e22) a benzothiazolyoxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and
(e23) a furanyl group;
(e24) a furanyl group having, on the ring, 1 to 3 substituting groups which may be the same or different and are selected from she group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e25) a thienyl group
(e26) a thienyl group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group,
(e27) a naphthyl group;
(e28) a naphthyl group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e29) a naphthyloxy group; and
(e30) a naphthyloxy group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group,
A is a nitrogen atom or a CZ group (wherein Z represents a hydrogen atom, (a) a halogen atom, (d) a (C₁-C₆) alkyl group, (e) a halo (C₁-C₆) alkyl group, (f) a (C₁-C₆) alkoxy group or (g) a halo (C₁-C₆) alkoxy group), and
Het represents 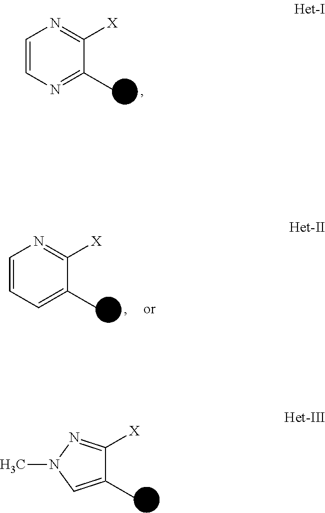
(wherein X represents a halogen atom or a halo (C₁-C₆) alkyl group, and the black circle represents a binding site)}, or
a salt thereof.

13. The endoparasite control agent according to claim 12, wherein
R¹ and R² are (a1) hydrogen atoms,
R³ and R⁴ may be the same or different, and are selected from the group consisting of
(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₃-C₆) cycloalkane,
Y¹ is a halogen atom;
Y² and Y⁴ are (d1) hydrogen atoms, and
Y³ is selected from the group consisting of
(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e6) a pyridyl group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e7) a pyridyloxy group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e9) a pyrimidyloxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e16) a quinolyloxy group having, on the ring, 1 to 6 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e17) a quinoxalyloxy group;
(e18) a quinoxalyloxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e19) a benzoxazolyloxy group;
(e20) a benzoxazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group.

14. The endoparasite control agent according to claim 12, wherein
R¹ and R² are (a1) hydrogen atoms,
R³ and R⁴ may be the same or different, and are selected from the group consisting of
(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,
Y¹ is (c1) a halogen atom,
Y² and Y⁴ are (d1) hydrogen atoms,
Y³ is selected from the group consisting of
(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (Ci-Cy) alkoxy group and (g) a halo (C₁-C₆) alkoxy group;
(e5) a pyridyl group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e14) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e17) a quinoxalyloxy group;
(e19) a benzoxazolyloxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and
Het is Het-I.

15. A method for controlling endoparasites, comprising orally or parenterally administering an effective amount of the endoparasite control agent according to claim 12 to a non-human mammal or a bird.

16. A method for controlling endoparasites, comprising orally or parenterally administering an effective amount of the endoparasite control agent according to claim 12 to a non-human mammal.

17. The method according to claim 15, wherein the non-human mammal is a domestic animal.

18. A pyrazine carboxamide derivative represented by the general formula (I): 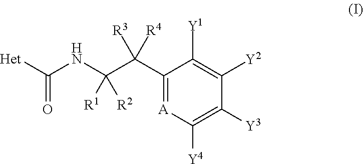
{wherein
R¹ and R² are (al) hydrogen atoms,
R³ and R⁴ may be the same or different, and are selected from the group consisting of
(b1) a hydrogen atom;
(b2) a (C₁-C₆) alkyl group; and
(b3) a (C₁-C₆) alkoxy group, or optionally
(b4) R³ and R⁴ together with the carbon atom bound to R³ and R⁴ form a (C₁-C₆) cycloalkane,
Y¹ is (c1) a halogen atom,
Y² and Y⁴ are (d1) hydrogen atoms,
Y³ is selected from the group consisting of
(e1) a phenyl group;
(e2) a phenyl group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e3) a phenoxy group;
(e4) a phenoxy group having, on the ring, 1 to 5 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e5) a pyridyl group;
(e8) a pyridyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e10) a pyrimidyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e11) a pyrazyloxy group;
(e12) a pyrazyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group;
(e13) a pyrazolyloxy group having, on the ring, 1 to 3 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group, (g) a halo (C₁-C₆) alkoxy group and (h) a formyl group;
(e15) a quinolyloxy group;
(e17) a quinoxalyloxy group;
(e19) a benzoxazolyloxy group;
(e21) a benzothiazolyloxy group; and
(e22) a benzothiazolyloxy group having, on the ring, 1 to 4 substituting groups which may be the same or different and are selected from the group consisting of (a) a halogen atom, (b) a cyano group, (c) a nitro group, (d) a (C₁-C₆) alkyl group, (e) a halo (C₁-C₆) alkyl group, (f) a (C₁-C₆) alkoxy group and (g) a halo (C₁-C₆) alkoxy group, and
Het represents the following formula Het-I: 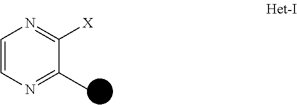
(wherein X represents a halogen atom or a halo (C₁-C₆) alkyl group, and the black circle represents a binding site)}, or
a salt thereof.

19. A method for controlling endoparasites, comprising orally or parenterally administering an effective amount of the endoparasite control agent according to claim 12 to a human.

20. The method according to claim 16, wherein the non-human mammal is a domestic animal.
